(19) United States
(12) Patent Application Publication
(10) Pub. No.: US 2009/0214605 A1
(43) Pub. Date: Aug. 27, 2009

(54) Hydrophilic Core Polymeric Micelles for the Delivery of Water-Soluble Compounds

(76) Inventors: Jean-Christophe Leroux, Montreal (CA); Marie-Christine Jones, Laval (CA); Pankaj Kumar Tewari, Edmonton (CA)

Correspondence Address: Klauber & Jackson, 411 Hackensack Ave., 4th Floor, Hackensack, NJ, 07601, US

(21) Appl. No.: 11/887,857

(22) PCT Filed: Apr. 5, 2006
(86) PCT No.: PCT/CA2006/000522

§ 371 Date: Jan. 16, 2009

Related U.S. Application Data
(60) Provisional application No. 60/668,097, filed on Apr. 5, 2005.

(51) Int. Cl. A61K 8/02; A61K 9/00; A61K 47/30; A61K 47/32; A61K 38/16; A61K 38/02; A61K 39/395; A61K 31/7052; A61K 31/715; A61P 43/00; A61Q 90/00

(52) U.S. Cl. 424/401; 424/400; 514/772.3; 514/772.4; 514/772.1; 514/12; 514/2; 424/130.1; 514/43; 514/44 R; 514/54

(57) ABSTRACT

The present invention describes the preparation of reverse polymeric micelles (RPM) through hydrophobically-modified star-shaped polyols. More specifically, RPM are obtained from polymeric structures where the polyol constitutes the hydrophilic domain while the hydrophobic shell is obtained either through the modification of existing chemical functions (e.g. esterification) or through copolymerization with hydrocarbon (alkyl)acrylate derivatives. Hydrophilic guests can be accommodated in the micelle core opening new avenues for the use of such carriers as delivery agents for polar active compounds.

### FIELD OF THE INVENTION

The invention relates to the field of chemical modification of star-shaped polyols in general and of star-shaped poly(glycerolmethacrylate) in particular, for the preparation of reverse polymeric micelles (RPM). RPM are core-shell structures characterized by a hydrophilic interior and hydrophobic exterior. Several synthetic routes can be employed to generate RPM differing in molecular weight, core density or hydrophobicity. From an application point of view, these RPM can be used to encapsulate polar guest molecules, which opens the door for their use as delivery systems for hydrophilic active compounds.

### BACKGROUND OF THE INVENTION

The absorption of hydrophilic molecules through physiological barriers such as the skin or the intestinal membrane is often restricted due to their low solubility in membrane constituents. For peptides/proteins, the oral bioavailability is further limited by enzymatic degradation. Accordingly, the administration of multiple doses is necessary in order to maintain therapeutic blood concentrations. Thus, such compounds may benefit from the development of suitable carriers. The present invention relates to the preparation of core-shell RPM from star-shaped polyols polymers and their use as carriers for polar active ingredients. The hydrophilic core should provide a protective environment for fragile molecules while the hydrophobic shell should promote the absorption and ultimately increase the bioavailability of the active ingredient.

Micelles are formed as a result of the association of amphiphilic molecules in a solvent that is selective for either moiety with the soluble moiety forming the micellar shell (Jones and Leroux, Eur. J. Pharm. Biopharm. (1999), 48, 101-111). Although micelles have been studied mainly as drug delivery systems for hydrophobic drugs, reverse micelles resulting from the association of amphiphilic molecules in apolar solvents, may represent a suitable vector for hydrophilic compounds. Structurally, reverse micelles are characterized by a hydrophilic interior where water-soluble compounds can be loaded, surrounded by a hydrophobic palisade which may aid in the absorption of the carrier's payload through interactions with biological membranes.

Reverse micelles were first developed using surface-active agents such as Aerosol OT (Silber et al. Adv. Coll. Int. Sci. (1999) 82, 189-252). In these systems, the presence of water is essential for micelle formation. Xu et al. (Macromolecules (2004) 37, 6264-6267) have described an amphiphilic core-shell polymer with complex hyperbranched-hyperbranched structures. They employed a one-pot two-step synthesis approach based on self-condensing (or multibranching) ring-opening polymerization of two monomers 3-ethyl-3-(hydroxymethyl)oxetane (hydrophobic) and glycidol (hydrophilic) in succession to generate the suprabranched macromolecules. Cho et al. (Macromolecules (2004) 37, 4227-4234) have studied the properties of RPM obtained from amphiphilic dendrimers. The micelles comprised a hydrophilic aliphatic polyether core and a hydrophobic docosyl exterior.

Oppenlaender et al., (U.S. Pat. No. 5,147,644, September 1992) have described a method for obtaining mixtures of polyglycerols fatty esters which can be used as emulsifiers in cosmetic and pharmaceutical formulations.

International patent application published under No. WO 03/047493 describes compositions and methods to promote transmucosal delivery of polar agents using reverse micelles. The reverse micelle composition necessarily includes a surfactant, a hydrophilic phase, a stabilizer to improve the stability of the micelles in the gastro-intestinal tract and one or more biologically active compound(s). Proposed compositions include reverse micelles of monoglycerides, diglycerides or fatty acids esters.

Ghosh et al. (Macromolecules (2003) 36, 9162-9169) have described amphiphilic poly(amidoamine) (PAMAM) dendrimers which can encapsulate water and polar guests. Third and fourth generation PAMAM dendrimers were modified with stearoyl acrylate. The RPM thus obtained showed the ability to extract hydrophilic compounds (acid red and copper(II) salts) from water into toluene.

Tomalia et al. (J. Mater. Chem. (1997) 7, 1199-1205) have prepared hydrophobically modified poly(amidoamine) (PAMAM) dendrimers. Second generation PAMAM dendrimers were modified using 1,2-epoxyhexane to generate RPM. Hydrophobically modified PAMAM were able to extract copper(II) salt from water into toluene.

Mecking et al. (Macromolecules (2000) 33, 3958-3960) have studied the ability of RPM to solubilize palladium salts in apolar solvents (toluene and chloroform). RPM were obtained from amphiphilic hyperbranched polyglycerol prepared by ring-opening multibranching polymerization followed by esterification of hydroxyl functions using palmitoyl chloride (C16). The RPM were used as catalysts in chemical reactions.

Dworak et al. (Polym. Bull. (2000) 50, 47-54) have described the synthesis of hydrophobically modified polyglycidol. Linear and comb-like polyglycidol were synthesized by living anionic polymerization. Hydrophilic polyglycidol was esterified using acetyl chloride at different grafting ratio to obtain a series of thermo-responsive polymers.

Sunder et al. (Angew. Chem. Int. Ed. (1999) 38(23), 3552-3555) have described the synthesis of reverse unimolecular micelles based on amphiphilic hyperbranched polyglycerol. The polymers were prepared by ring-opening multibranching polymerization followed by esterification of hydroxyl functions using acid chloride derivatives of fatty acids. These micelles have shown the ability to extract a hydrophilic dye (Congo red) from water into chloroform. RPM were also obtained from multi-arm block copolymers of glycerol and methylacrylate (Maier et al., Macromol. Rapid Comm. (2000) 21, 226-230). In this instance, hyperbranched polyglycerols were used as multifunctional initiators for the atom transfer radical polymerization (ATRP) of methyl acrylate. The RPM consisted of a polyether interior and poly(methyl acrylate) arms. In another study (Kramer et al., Angew. Chem. Int. Ed. (2002) 41, 4252-4256), pH-sensitive RPM were described where hyperbranched polyglycerol and polyethyleneimine were used as scaffolds. pH-sensitive ketal and imine bonds were used to introduce the hydrophobic fatty acids shell. The loading and release of Congo red (a hydrophilic dye) was studied as a function of pH. Amphiphilic poly(ethylenimine)s have also shown the ability to quantitatively extract various dyes from an aqueous phase into apolar media (Chen et al., Macromolecules (2005) 38, 227-229) while poly(glycerol)s were able to extract catalytically active polar pincer Pt(II) complexes (Stiriba et al., J. Am. Chem. Soc. (2002) 124, 9698-9699; Slagt et al., Macromolecules (2002) 35, 5734-5737).

None of the systems described above have used star-shaped polymers as scaffolds for the preparation of RPM. In contrast to dendrimers, star-shaped polymers are easily synthesized, often in a single step, using controlled/living polymerization procedures such as atom transfer radical polymerization (ATRP) or anionic polymerization. Also, the number of arms is controlled by varying the multifunctional initiator, as opposed to hyperbranched structures where the actual number of arms cannot be predicted. The ability of RPM to extract hydrophilic molecules from water is determined mainly by the polarity of the core and the presence of chemical functions able to participate in hydrogen bonding with the encapsulated guest. Star-shaped polyols are characterized by a high density of hydroxyl function within the core, contrary to the hyperbranched polyglycidol structures described previously where the hydroxyl functions are concentrated at the periphery.

A need therefore exists to formulate RPM using star-shaped polymers which would overcome the drawbacks of micelles prepared using hyperbranched or dendritic structures.

### SUMMARY OF THE INVENTION

The present work focuses on the development of RPM from amphiphilic star-shaped polyols to be used as delivery systems for water-soluble active ingredients. The hydrophilic micellar core may be prepared from the polymerization of any hydroxyl bearing monomers including glycerol (alkyl)acrylate, hydroxyl(alkyl)(alkyl)acrylate or acrylate derivatives of sugars such as 2-methacryloxyethyl glucoside, d-gluconamidoethyl methacrylate, 2-lactobioamidoethyl methacrylate. Star-shaped poly(glycerol methacrylate) can also be obtained from the polymerization and hydrolysis of an epoxide-bearing precursor (i.e glycidyl methacrylate). Two approaches have been preferably selected for the synthesis of such RPM.

The first method is based on the synthesis of star-shaped polyols by living/controlled polymerization using a multifunctional initiator. The hydrophobic shell is then introduced by reaction of the hydroxyl groups with some electrophiles. In fact, the polyol is grafted with some reactants. In particular, the hydroxyl groups can react with electrophiles such as acylating agents or alkylating agents so as to form the desired hydrophobic shell. The acylating agent can be, in a non-limitative manner, an acyl halide (preferably acyl chloride), an anhydride, a carboxylic acid or a derivative thereof such as an activated carboxylic acid. The alkylating agent can be an alkyl halide or any alkyl chain having a suitable leaving group.

In the second approach, diblock copolymers of a hydroxyl-bearing monomer and an alkyl (alkyl)acrylate derivative are synthesized by living/controlled polymerization using a multifunctional initiator. The aim is to generate hydrophilic and hydrophobic segments of controlled length.

RPM thus obtained are soluble in apolar solvents and may be formulated in pharmaceutical oils for the delivery of hydrophilic active compounds, including but not limited to therapeutic peptides/proteins. They may find applications in oral, parenteral (subcutaneous, intramuscular and intraperitoneal) or transdermal delivery of active ingredients. In all instances, loading the guest molecule inside the RPM may favor its permeation through physiological barriers and, in the case of peptides/proteins, may afford protection against degradation.

In accordance with one aspect of the present invention, there is provided a reverse micelle composition comprising:

a) a reverse micelle comprising a hydrophilic core consisting of a star-shaped polyol, and a hydrophobic shell comprising alkyl chains, the alkyl chains being the same or different; and

b) a water-soluble active agent contained in the hydrophilic core.

In accordance with another aspect of the present invention, there is provided a reverse micelle composition comprising:
a reverse micelle comprising a star-shaped polyol at least partially grafted with alkyl chains or acyl chains, the alkyl chains being the same or different and the acyl chains being the same or different, wherein an inner portion of the star-shaped polyol comprising free hydroxyl groups defines a hydrophilic core, and wherein an outer portion of the star-shaped polyol comprising the alkyl chains or the acyl chains defines a hydrophobic shell; and
a hydrophilic active agent contained in the hydrophilic core.

In accordance with another aspect of the present invention, there is provided a process for preparing a reverse micelle composition as defined in the present invention. The process comprises mixing together the hydrophilic active agent and a composition comprising a micelle as defined in the present invention and a hydrophobic pharmaceutically acceptable vehicle

In accordance with another aspect of the present invention, there is provided a process for preparing a reverse micelle composition as defined in the present invention. The process comprises mixing together a first composition comprising a hydrophilic active agent and a polar solvent, and a second composition comprising a micelle as defined in the present invention and a hydrophobic pharmaceutically acceptable vehicle.

In accordance with another aspect of the present invention, there is provided the use of a reverse micelle comprising a hydrophilic core consisting of a star-shaped polyol; and a hydrophobic shell consisting of alkyl chains, the alkyl chains being the same or different, as a delivery system for a hydrophilic active agent.

In accordance with another aspect of the present invention, there is provided the use of a reverse micelle comprising a hydrophilic core consisting of a star-shaped polyol; and a hydrophobic shell consisting of alkyl chains, the alkyl chains being the same or different, in the manufacture of a medicament comprising a hydrophilic active agent.

In accordance with another aspect of the present invention, there is provided the use of a reverse micelle comprising a star-shaped polyol grafted with alkyl chains or acyl chains, the alkyl chains being the same or different and the acyl chains being the same or different, wherein an inner portion of the star-shaped polyol comprising free hydroxyl groups defines a hydrophilic core, and wherein an outer portion of the star-shaped polyol comprising the alkyl chains or the acyl chains defines a hydrophobic shell, as a delivery system for a hydrophilic active agent.

In accordance with another aspect of the present invention, there is provided the use of a reverse micelle comprising a star-shaped polyol grafted with alkyl chains or acyl chains, the alkyl chains being the same or different and the acyl chains being the same or different, wherein an inner portion of the star-shaped polyol comprising free hydroxyl groups defines a hydrophilic core, and wherein an outer portion of the star-shaped polyol comprising the alkyl chains or the acyl chains defines a hydrophobic shell, in the manufacture of a medicament comprising a hydrophilic active agent.

In accordance with another aspect of the present invention, there is provided a delivery system for a hydrophilic active agent, the system comprising a hydrophilic core consisting of a star-shaped polyol and being adapted to receive the hydrophilic active agent; and a hydrophobic shell consisting of alkyl chains, the alkyl chains being the same or different.

In accordance with another aspect of the present invention, there is provided a delivery system for a hydrophilic active agent, the system comprising a star-shaped polyol at least partially grafted with alkyl chains or acyl chains, the alkyl chains being the same or different and the acyl chains being the same or different, wherein an inner portion of the star-shaped polyol comprises free hydroxyl groups defining a hydrophilic core, and wherein an outer portion of the star-shaped polyol comprises the alkyl chains or the acyl chains defining a hydrophobic shell.

In accordance with another aspect of the present invention, there is provided a method for using a reverse micelle, the method comprising inserting a hydrophilic active agent in the reverse micelle comprising a hydrophilic core consisting of a star-shaped polyol; and a hydrophobic shell consisting of alkyl chains, the alkyl chains being the same or different, so as to deliver the hydrophilic active agent.

In accordance with another aspect of the present invention, there is provided a method for using a reverse micelle, the method comprising inserting a hydrophilic active agent in the reverse micelle hydrophilic core consisting of a star-shaped polyol; and a hydrophobic shell consisting of alkyl chains, the alkyl chains being the same or different, thereby obtaining a medicament for delivering the hydrophilic active agent contained therein.

In accordance with another aspect of the present invention, there is provided a method for using a reverse micelle, the method comprising inserting a hydrophilic active agent in the reverse micelle comprising a star-shaped polyol grafted with alkyl chains or acyl chains, the alkyl chains being the same or different and the acyl chains being the same or different, wherein an inner portion of the star-shaped polyol comprising free hydroxyl groups defines a hydrophilic core, and wherein an outer portion of the star-shaped polyol comprising the alkyl chains or the acyl chains defines a hydrophobic shell, so as to deliver the hydrophilic active agent.

In accordance with another aspect of the present invention, there is provided a method for using a reverse micelle, the method comprising inserting a hydrophilic active agent in the reverse micelle comprising a star-shaped polyol grafted with alkyl chains or acyl chains, the alkyl chains being the same or different and the acyl chains being the same or different, wherein an inner portion of the star-shaped polyol comprising free hydroxyl groups defines a hydrophilic core, and wherein an outer portion of the star-shaped polyol comprising the alkyl chains or the acyl chains defines a hydrophobic shell, thereby obtaining a medicament for delivering the hydrophilic active agent contained therein.

In accordance with a preferred embodiment of the present invention, the star-shaped polyol is selected from the group consisting of poly(glycerol (alkyl)acrylate), poly(hydroxy(alkyl)(alkyl)acrylate), poly(>methylglucoside (alkyl)acrylate), poly(2-gluconamidoethyl (alkyl)acrylate) and poly(2-lactobionamidoethyl (alkyl)acrylate), etc.

In accordance with a preferred embodiment of the present invention, the polyol is a copolymer of hydroxyl-bearing (alkyl)acrylate and an ionisable (alkyl)acrylate derivative, such as an (alkyl)acrylic acid (i.e. methacrylic acid) or an (alkyl)amino(alkyl)acrylate (i.e. aminoethylmethacrylate). The hydroxyl-bearing (alkyl)acrylate is selected from the group consisting of glycerol (alkyl)acrylate, hydroxy(alkyl) (alkyl)acrylate, β-methylglucoside (alkyl)acrylate, 2-gluconamidoethyl (alkyl)acrylate and 2-lactobionamidoethyl (alkyl)acrylate, etc.

In accordance with a preferred embodiment of the present invention, the star-shaped polymer is formed using a multifunctional initiator bearing from 3 to 20 initiating arms, preferably 3, 4, 6 or 10 arms, and more preferably 4 arms.

In accordance with a preferred embodiment of the present invention, the alkyl chain is linked to the star-shaped poly(glycerol (alkyl)acrylate) core by a covalent bond.

In accordance with a preferred embodiment of the present invention, the alkyl chain is linked to the star-shaped poly(glycerol (alkyl)acrylate) core by a non-hydrolysable bond.

In accordance with a preferred embodiment of the present invention, the alkyl chain is linked to the star-shaped poly(glycerol (alkyl)acrylate) core by a hydrolysable bond including but not limited to ester, ketal or anhydride bond.

In accordance with a preferred embodiment of the present invention, the star-shaped polyol, excluding the grafting chains, has a molecular weight between 3000 and 200000 g/mol.

In accordance with a preferred embodiment of the present invention, the active agent is selected from the group consisting of cytokines, peptidomimetics, peptides, proteins, toxoids, antibodies, nucleosides, nucleotides, nucleic acids, polysaccharides, analgesics and anti-inflammatory agents, anthelmintics, anti-arrhythmic agents, anti-asthma agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents and immunosuppressants, anti-protozoal agents, anti-thyroid agents, anti-tussives, anxiolytic, sedatives, hypnotics and neuroleptics, β-blockers, cardiac inotropic agents, diuretics, anti-parkinsonian agents, gastrointestinal agents, histamine H,-receptor antagonists, keratolytics, lipid regulating agents, muscle relaxants, anti-anginal agents, nutritional agents, analgesics, sex hormones, stimulants, and cytokines.

In accordance with a preferred embodiment of the present invention, there is provided a pharmaceutical composition which comprises a reverse micelle composition of the present invention dissolved in a pharmaceutically acceptable oil and administered orally, topically or parentally.

In accordance with a preferred embodiment of the present invention, there is provided a cosmetic preparation, which comprises a reverse micelle composition of the present invention dissolved in a pharmaceutically acceptable oil.

In accordance with a preferred embodiment of the present invention, there is provided a process for preparing the pharmaceutical composition of the present invention, which comprises the dissolution of a hydrophilic active ingredient and an amphiphilic star-shaped polymer in an oleaginous vehicle, with or without surface active agents.

In accordance with a preferred embodiment of the present invention, there is provided a process for preparing the cosmetic preparation of the present invention, which comprises: the dissolution of a hydrophilic active agent and an amphiphilic star-shaped polymer in an oleaginous vehicle, with or without surface active agents.

In accordance with a preferred embodiment of the present invention, the hydrophobic pharmaceutically acceptable vehicle can be a pharmaceutically acceptable oil.

In accordance with a preferred embodiment of the present invention, the hydrophilic active agent, the micelle and the pharmaceutically acceptable vehicle are mixed together and heated. They can be heated at a temperature of about 30° C. to about 100° C. and preferably of about 35° C. to about 60° C.

In accordance with a preferred embodiment of the present invention, the hydrophilic active agent, the polar solvent, the micelle and the pharmaceutically acceptable vehicle are mixed together and heated. They can be heated at a temperature of about 30° C. to about 100° C. and preferably of about 35° C. to about 60° C. The process can further comprise separating the polar solvent from the rest of the micelle composition. The polar solvent can be removed from the micelle composition by evaporating it.

In accordance with a preferred embodiment of the present invention, each of the alkyl chains is linked to the star-shaped polyol core by means of a covalent bond. The covalent bond can be a non-hydrolysable bond or a hydrolysable bond. The non-hydrolysable bond can be an ether bond. Each of the alkyl chains can be directly connected to a hydroxyl group of the polyol through the ether bond. Each of the alkyl chains can be linked to the star-shaped core via a hydrolysable bond. The hydrolysable bond can be an ester bond, a ketal bond or an anhydride bond. An ester bond is particularly preferred. Each of the alkyl chains can be linked to a hydroxyl group of the star-shaped core by means of the carbonyl of the ester bond.

In accordance with a preferred embodiment of the present invention, the alkyl chains can be the same and they each comprise from 4 to 30 carbon atoms and preferably from 8 to 20 carbon atoms. The alkyl chains can be linear or branched, and saturated or unsaturated. The unsaturated alkyl chains can be monounsaturated or polyunsaturated.

In accordance with a preferred embodiment of the present invention, the acyl chains can be the same and they each comprise from 4 to 30 carbon atoms and preferably from 8 to 20 carbon atoms. The acyl chains can be linear or branched, and saturated or unsaturated. The unsaturated acyl chains can be monounsaturated or polyunsaturated.

In accordance with a preferred embodiment of the present invention, the term alkyl, when referring to the expressions (alkyl)acrylate, (alkyl)amino, (alkyl)acrylic, hydroxy(alkyl), alkyl halide, alkyl chain or the like, refers to an alkyl chain having 1 to 30 carbon atoms. It can also refer to an alkyl chain having 1 to 20 carbon atoms. Such alkyls can be linear or branched, and saturated or unsaturated (monounsaturated or polyunsaturated).

In accordance with a preferred embodiment of the present invention, the unsaturated alkyl chains may be monounsaturated or polyunsaturated.

In accordance with a preferred embodiment of the present invention, at least 10% of the hydroxyls groups of the polyol are grafted with the acyl chains or the alkyl chains.

In accordance with a preferred embodiment of the present invention, at least 20% of the hydroxyls groups of the polyol are grafted with the acyl chains or the alkyl chains.

In accordance with a preferred embodiment of the present invention, at least 40% of the hydroxyls groups of the polyol are grafted with the acyl chains or the alkyl chains.

In accordance with a preferred embodiment of the present invention, at least 50% of the hydroxyls groups of the polyol are grafted with the acyl chains or the alkyl chains.

In accordance with a preferred embodiment of the present invention, at least 60% of the hydroxyls groups of the polyol are grafted with the acyl chains or the alkyl chains.

In accordance with a preferred embodiment of the present invention, about 40% to about 70% of the hydroxyls groups of the polyol are grafted with the acyl chains or the alkyl chains.

For the purpose of the present invention, the following terms are defined below.

Homopolymer consists of polymeric chains made up of identical repeating units.

Block copolymer consists of polymeric chains made up of two or more homopolymer blocks, each homopolymer being different in nature.

Hyperbranched polymers are tree-like polymeric structures based on the repetition of a monomeric unit with divergent points. Hyperbranched polymers are closely related to dendrimers but are less regular since they are synthesized in a single step using multifunctional monomers. Recent researches have shown that the amphiphilic core-shell polymers with complex hyperbranched-hyperbranched structures can be developed. These core-shell hybrids resulting from hyperbranched macromolecules are also termed as suprabranched polymers.

Star-shaped polymers present multi-arm structure with each arm emanating from a central focal point or a concentric region. Star-shaped polymers can be prepared by a convergent or divergent approach. In the former, linear polymer chains are first synthesized and then cross-linked using a cross-linking agent. In the latter method, polymer chains are grown from a multifunctional initiator.

The active agent to be incorporated into the delivery system is water-soluble. The water-soluble active agent can be any biologically active, preferably therapeutic material, particularly water-soluble proteins, peptides and other pharmaceutically-active compounds, i.e., drugs, and compounds which may have use as diagnostic agents. Vitamins and other food supplements are also within the definition of the active agent.

The agents have molecular weights greater than about 100 g/mol, preferably greater than 300 g/mol and more preferably greater than 400 g/mol. Suitable agents are also characterized by poor absorption through the GI tract with oral bioavailabilities (compared to i.v. availabilities) of preferably less than about 50%, more preferably less than about 35% and most preferably less than about 20% when administered at therapeutic dosage levels.

By “therapeutic” is meant an amount of the agent that produces the usual and desired pharmacological or physiological response to that agent elicited when it is administered by parenteral routes. The amount of active material to be administered to be “therapeutic” will be easily determined by those skilled in the art based upon the concentration and the repetition of the dosage.

Hydrophilic therapeutic agents suitable for use in the pharmaceutical systems and methods of the present invention are not particularly limited. Suitable hydrophilic therapeutic agents include hydrophilic drugs (i.e., conventional non-peptidic drugs), hydrophilic macromolecules such as cytokines, peptidomimetics, peptides, proteins, toxoids, antibodies, nucleosides, nucleotides, nucleic acids and genetic material, and other hydrophilic compounds, such as polysaccharides. The aqueous solubility of the hydrophilic therapeutic agent should be greater than about 1 mg/mL

Chemical classes of suitable therapeutic agents include the water-soluble proteins or peptides. One group of agents are water-soluble peptides having a molecular weight from about 300 to about 2,000 g/mol and containing at least one and preferably two or more peptide bonds. A second group of agents are water-soluble polypeptides from about 2,000 to about 10,000 g/mol having at least three and preferably five or more peptide bonds. A third group of agents are water soluble proteins having molecular weights greater than 10,000 g/mol and containing at least six and preferably ten or more peptide bonds.

The final formulation is obtained as a solution of drug-loaded RPM in a pharmaceutical oil. Optionally, the hydrophilic therapeutic agent can be present in a first, solubilized amount, and a second, non-solubilized (suspended) amount. Surface active ingredients may be added to the formulation in order to aid the dispersion of the oil phase in aqueous media such as the GI fluids; these may include sorbitan fatty acid esters, poly(ethylene glycol) sorbitan fatty acid esters, polyoxyethylene-polyoxypropylene block copolymers, polyethylene glycol alkyl ether. The hydrophilic agents can be any agent having therapeutic or other value when administered to an animal, particularly to a mammal, such as drugs, nutrients, cosmetics (cosmeceuticals), and diagnostic agents. It should be understood that while the invention is described with particular reference to its value for oral dosage forms, the invention is not so limited. Thus, hydrophilic drugs, nutrients, cosmeceuticals and diagnostic agents which derive their therapeutic or other value from, for example, transmembrane (transport across a membrane barrier of therapeutic significance), nasal, buccal, rectal, vaginal or pulmonary administration are still considered to be suitable for use in the present invention.

Specific non-limiting examples of therapeutic agents that can be used in the pharmaceutical compositions of the present invention include analgesics and anti-inflammatory agents, anthelmintics, anti-arrhythmic agents, anti-asthma agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents and immunosuppressants, anti-protozoal agents, anti-thyroid agents, anti-tussives, anxiolytic, sedatives, hypnotics and neuroleptics, β-blockers, cardiac inotropic agents, diuretics, anti-parkinsonian agents, gastrointestinal agents, histamine H,-receptor antagonists, keratolytics, lipid regulating agents, muscle relaxants, anti-anginal agents, nutritional agents, analgesics, sex hormones, stimulants, cytokines, peptidomimetics, peptides, proteins, toxoids, antibodies, nucleosides, nucleotides, genetic material, and nucleic acids.

Suitable therapeutic peptides of molecular weight 300 to 2,000 g/mol having 3 to 10 amino acid moieties include: fibrinogen receptor antagonist peptides, RGD containing peptides, which are tetrapeptides of average molecular weight of about 600 g/mol, having the amino acids arginine-glycine-aspartic acid, in that order, as part of their sequence with the fourth position of the tetrapeptide variable. Such peptides are highly potent platelet aggregation inhibitors active at plasma concentrations as low as 1 μmol/mL. Fibrinogen antagonists are described in published applications EP 0 341 915 and EP 0 423 212 and EP Application No. 9031 1537.6 whose disclosures are herein incorporated by reference in their entirety. RGD-containing peptides and peptide-like molecules are generally present in amounts ranging from about 10 mg to about 500 mg per gram of the drug delivery composition depending on the solubility and therapeutic potency of the compound.

Other fibrinogen antagonists useful in the present invention are those peptides disclosed in Pierschbacher et al., WO 89/05150 (U.S. Pat. No. 8,804,403); Marguerie, EP 0 275 748; Adams et al., U.S. Pat. No. 4,857,508; Zimmerman et al., U.S. Pat. No. 4,683,291; Nutt et al., EP 0 410 537; Nutt et al., EP 0 410 539; Nutt et al, EP 0 410 540; Nutt et al., EP 0 410 541; Nutt et al., EP 0 410 767; Nutt et al., EP 0 410 833; Nutt et al., EP 0 422 937; Nutt et al., EP 0 422 938; AHg et al., EP 0 372 486 Ohba et al., WO 90/02751 (PCT/JP89/00926); Klein et al., U.S. Pat. No. 4,952,562; Scarborough et al., WO 90/15620 (PCT/US90/03417); Ali et al., PCT US90/06514, filed Nov. 2, 1990; peptide like compounds as disclosed in ANg et al., EP 0 381 033; and AHg et al., EP 0 384 362, the disclosures of all of these being incorporated herein in their entireties by reference.

Another useful class of peptides are hexapeptides related to growth hormone releasing peptide (GHRP) (C. Y. Bowers, J. Pediatr. Endocrinol., 6, 21, 1993, incorporated herein in its entirety by reference). Growth hormone releasing peptides are disclosed, for instance, in Momany, U.S. Pat. No. 4,411,890; Momany, U.S. Pat. No. 4,410,513; Momany, U.S. Pat. No. 4,410,512; Momany, U.S. Pat. No. 4,228,158; Momany, U.S. Pat. No. 4,228,157; Momany U.S. Pat. No. 4,228,156; Momany, U.S. Pat. No. 4,228,155; Momany, U.S. Pat. No. 4,226,857; Momany U.S. Pat. No. 4,224,316, Momany U.S. Pat. No. 4,223,021; Momany, U.S. Pat. No. 4,223,020; Momany, U.S. Pat. No. 4,223,019; Bowers et al., U.S. Pat. No. 4,880,778; Bowers et al., U.S. Pat. No. 4,880,777; Bowers et al., U.S. Pat. No. 4,839,344; Bowers et al., U.S. Pat. No. WO 89/10933 (PCT/US89/01829); Bowers et al., EP-A 398 961, Bowers et al. EP-A 400 051, all of which are fully incorporated herein by reference. These compounds are useful for accelerating the growth of humans and animals.

Antagonists of GHRP are useful in clinical situations where abnormally accelerated growth or excessive plasma levels of growth hormone are encountered. Both agonists and antagonists of GHRP are usefully present in the range of 0.001 to 100 mg per gram of drug composition, depending on their potency.

Nonapeptidyl vasopressin Vi and V₂ receptor agonists and antagonists are used clinically to treat conditions of excessive urinary output and blood clotting factor VIII deficiency. Particularly useful agents include arginine vasopressin (AVP), lysine vasopressin (LVP) and desmopressin (dDAVP), with molecular weights 1084, 1056 and 1069 g/mol respectively.

Yet another class of useful peptides include luteinizing hormone-releasing hormone (LH-RH) and its analogues. These peptides contain about 10 natural or synthetically produced amino acid residues and have molecular weights ranging from about 1,000 to about 1,600 g/mol. Suitable examples include LH-RH itself, with a sequence of pGlu-His-Trp-Ser-Tyr-Gly-Leu-Arg-PrO-Gly-NH₂ (MW=1,182 g/mol) where pGlu stands for a pyroglutamic acid residue and the C-terminus of the peptide is amidated (—NH₂); the LH-RH agonist analogue, Des-Glyio, D-Trp₆, PrO₉-LH-RH, ethylamide (MW=1,283 g/mol).

Melanocyte stimulating hormones (MSHs) and analogues, having molecular weights ranging from about 800 to about 3,000 g/mol, may also be usefully incorporated into these formulations. Particularly preferred are analogues displaying prolonged circulatory half-lives and/or increased resistance to proteolytic degradation.

Peptidyl proteinase inhibitors are another category of peptides and peptide analogues that may be usefully incorporated into the drug composition. Particularly preferred are: inhibitors of metalloproteinases, such as collagenase and elastase, which are useful in treating certain metastatic cancers and certain inflammatory diseases, such as arthritis; inhibitors of proteases coded on viral genomes, such as the HIV-1 and HIV-2 viral proteases; inhibitors of angiotensin converting enzyme (ACE inhibitors) or of renin, useful in the treatment of hypertension; and inhibitors of blood clotting cascades proteases, such as thrombin inhibitors, useful for treating thrombosis. Also, useful as anticoagulants are peptides and polypeptide fragments of the leech protein hirudin, as well as analogues of these fragments and hirudin itself.

Calcitonins, such as those set forth in U.S. Pat. No. 5,002,771 which is incorporated herein in its entirety, are a set of therapeutic polypeptides useful for treating hypercalcemia and bone loss. Preferred calcitonins for incorporation into the formulation are salmon, eel and human calcitonins. Salmon calcitonin and eel calcitonin are most preferred because of their higher potency and more favorable pharmacokinetic profile. Human calcitonin is preferred in those patients where adverse reactions or insensitivity to the others is known or suspected. Synthetic salmon, eel or human calcitonins have the same amino acid sequence as their naturally occurring counterparts, but may, in some cases, be truncated or chemically altered versions of the natural molecule. Their molecular weights range from about 3,300 to 3,500 g/mol.

Other polypeptide regulators of calcium metabolism which could be usefully included in the formulation include the 84 amino acid residue polypeptides human or bovine parathyroid hormone (PTH) with molecular weights of 9,425 and 9,51 1 g/mol, respectively, and truncated versions and biologically active fragments thereof having 14 to 83 amino acid residues and molecular weights from 1,400 to 9,950 g/mol. Also useful in this category are PTH-related peptides, such as the human hypercalcemia of malignancy peptide which has 86 amino acid residues and a molecular weight of 9,903 g/mol. Biologically active fragments of this molecule having 14 to 85 amino acid residues and molecular weights from about 1,400 to about 9,950 g/mol may also be usefully included in the formulation, as well as polypeptide analogues of such fragments having agonist or antagonist activities.

Atrial natriuretic peptides (ANPs) and their analogues are polypeptides useful for treatment of hypertension. Particularly preferred for use in these formulations are human ANPs and their analogues with molecular weights of from about 1,000 to about 4,000 g/mol. Brain natriuretic peptides are also useful for this purpose.

The insulins are another group of polypeptides which may be incorporated into the formulation. Human, bovine, porcine or ovine insulins or chemically modified derivatives thereof would be particularly preferred. The insulins are disulfide-linked, dimeric polypeptides having two distinct chains, A and B, and a molecular weight of about 6,000 g/mol for the dimeric molecule.

Other useful polypeptides in this molecular weight range include amylin, insulin-like growth factors I, II and III (IGF-I, IGF-II, IGF-III), somatomedins, epidermal growth factor (EGF), and transforming growth factor-α (TGF-α). Polypeptide analogues of these molecules may also be incorporated into the formulation.

Proteins useful for incorporation into these formulations include: human, bovine, ovine or porcine growth hormone; α-, β-, or γ-interferons; lymphokines, such as interleukins 1 to 6; growth factors, such as platelet-derived growth factor, acidic or basic fibroblast growth factor; therapeutic enzymes, such as asparaginase or superoxide dismutase; erythropoietins; and monoclonal antibodies or their antigen-binding fragments.

Further, suitable agents include water soluble complex polysaccharides having at least two and preferably three or more monosaccharide units and additionally containing one or more of the following chemical substituents: amino groups (free or acylated), carboxyl groups (free or acylated), phosphate groups (free or esterified) or sulfate groups (free or esterified).

Particularly preferred polysaccharides include heparins, useful as anticoagulants, and polysaccharide inhibitors of the mammalian cell lectins, known collectively as ‘selectins’, useful as anti-inflammatory agents.

Also, suitable agents include nucleosides, nucleotides and their polymers. Suitable nucleosides include 3′-azido-3′-deoxythimidine, 2′,3′-dideoxy-derivatives of adenosine, cytidine, inosine, thymidine or guanosine. Suitable polynucleotides include “anti-sense” nucleotides having 3 to 30 nucleotide bases with nucleotide sequences complimentary to those coding for viral proteins or RNA's, oncogene proteins or RNA's, or inflammatory proteins or RNA's. Also useful are polynucleotides having 3 to 30 bases capable of forming triple helix structures with the DNA coding for the above.

Preferred water soluble active agents include RGD fibrinogen receptor antagonists, enkephalins, growth hormone releasing peptides and analogues, vasopressins, desmopressin, luteinizing hormone releasing hormones, melanocyte stimulating hormones and analogues, calcitonins, parathyroid hormone, PTH-related peptides, insulins, atrial natriuretic peptides and analogues, growth hormones, interferons, lymphokines, erythropoietins, interleukins, colony stimulating factors, tissue plasminogen activators, tumor necrosis factors, complex polysaccharides, and nucleosides, nucleotides and their polymers.

In one embodiment, the hydrophilic therapeutic agent is a nutritional agent.

In another embodiment, the hydrophilic therapeutic agent is a cosmeceutical agent.

In another embodiment, the hydrophilic therapeutic agent is a diagnostic agent.

Although the invention is not limited thereby, examples of hydrophilic therapeutic agents suitable for use in the compositions and methods of the present invention include the following preferred compounds, as well as their pharmaceutically acceptable salts, isomers, esters, ethers and other derivatives:

acyclovir sodium; acetyl cysteine; acetylcholine chloride; alendronate; alglucerase; amantadine hydrochloride; ambenomium; amifostine; aminocaproic acid; antihemophilic factor (human); antihemophilic factor (porcine); antihemophilic factor (recombinant); aprotinin; asparaginase; bacitracin; bleomycin sulfate; calcitonin human; calcitonin salmon; carboplatin; capecitabine; capreomycin sulfate; cefazolin sodium; cefoperazone; cefotetan disodium; cefoxitin sodium; ceftriaxone; cephapirin sodium; chorionic gonadotropin; cidofovir; clidinium bromide; clindamycin and clindamycin derivatives; clondronate; colistimethate sodium; colistin sulfate; corticotropin; cromolyn sodium; cytarabine; daltaperin sodium; danaparoid; deferoxamine; denileukin diftitox; desmopressin; diatrizoate meglumine and diatrizoate sodium; dicyclomine; didanosine; dopamine hydrochloride; dornase alpha; doxacurium chloride; editronate disodium; enkephalin; enoxaprin sodium; ephedrine; esmolol hydrochloride; factor IX; famciclovir; fluoxetine hydrochloride; ganciclovir; granulocyte colony stimulating factor; granulocyte-macrophage stimulating factor; gentamycin; glycopyrolate; gonadotropin releasing hormone and synthetic analogs thereof; goserelin; interleukin-2; interleukin-3; insulin; isofosfamide; lamivudine; leucovorin calcium; leuprolide acetate; mannitol; menotropins; methenamine; methscopolamine; metformin hydrochloride; metroprolol; meziocillin sodium; nafarelin; nedocromil sodium; neostigmine bromide; neostigmine methyl sulfate; octreotide acetate; oxytocin; pamidronate disodium; pancuronium bromide; pentamindine isethionate; pentoxifylline; phentolamine mesylate; phenylalanine; platelet derived growth factor-human; polymixin B sulfate; pralidoxime chloride; pramLintide; pyridostigmine bromide; ribavarin; somatostatin; spectinomycin; stavudine; streptokinase; streptozocin; tacrine hydrochloride; terbutaline sulfate; thiopental sodium; ticarcillin; tiludronate; timolol hydrogen maleate; tissue type plasminogen activator; D-glucuronate; tubocurarine chloride; urea; urokinase; vancomycin hydrochloride; valaciclovir hydrochloride; vasopressin and vasopessin derivatives; vecurnium bromide; vincristine sulfate; vinorelbine ditartrate; vitamin B12; warfarin sodium; zalcitabine; zolandronate; zidovudine; botulinum toxins (Botox) and glatiramer acetate (Copaxone).

Of course, salts, metabolic precursors, derivatives and mixtures of therapeutic agents may also be used where desired.

### BRIEF DESCRIPTION OF THE DRAWINGS

Further features and advantages of the invention will become more readily apparent from the following description of preferred embodiments as illustrated by way of examples in the appended drawings wherein:

FIG. 1 illustrates ¹H-NMR spectrum of poly(glycidylmethacrylate): (PGMA-io₆) (numeral suffix denotes number of monomers in the polymer chain) in CDCl₃.

FIG. 2 illustrates ¹H-NMR spectrum of poly(glycerol-methacrylate): (PG_{O}HMA₁₀₆) (numeral suffix denotes number of monomers in the polymer chain) in DMSO-d₆.

FIG. 3 illustrates AFM image of poly(glycerolmethacrylate) at molecular level. (Mn=150,000 g/mol).

FIG. 4 illustrates ¹H-NMR spectrum of the 50% stearoyl group substituted poly(glycerolmethacrylate): P(GO_{H}MA₇₅-C18) (grafting ratio 50%) in CDCl₃.

FIG. 5 illustrates ¹H-NMR spectrum of star-shaped poly(glycidylmethacrylate)-b-poly(laurylmethacrylate): PGMA-/>PLMA (75:25) in CDCl₃.

FIG. 6 illustrates ¹H-NMR spectrum of the poly(glycerolmethacrylate)-/?-poly(laurylmethacrylate): PGO_{H}MA-£>>-PLMA (75:25) in pyridine-ds.

FIG. 7 illustrates the extraction of a hydrophilic dye (Congo Red) from water (top layer) into methylene chloride (bottom layer) using PG_{O}HMA-C 16 (grafting ratio 25%).

### DETAILED DESCRIPTION OF THE INVENTION

The goal of the present invention is to develop an RPM formulation from hydrophobically-modified star-shaped polyols such as poly(glycerolmethacrylate). Star-shaped poly(glycerolmethacrylate) can be polymerized from a wide variety of multifunctional initiators bearing 3 to 20 arms (Robello et al., Macromolecules (2002) 35, 9334-9344 and Matyjaszewski et al., Macromolecules (1999) 32, 6526-6535). Examples of suitable initiators are shown below:

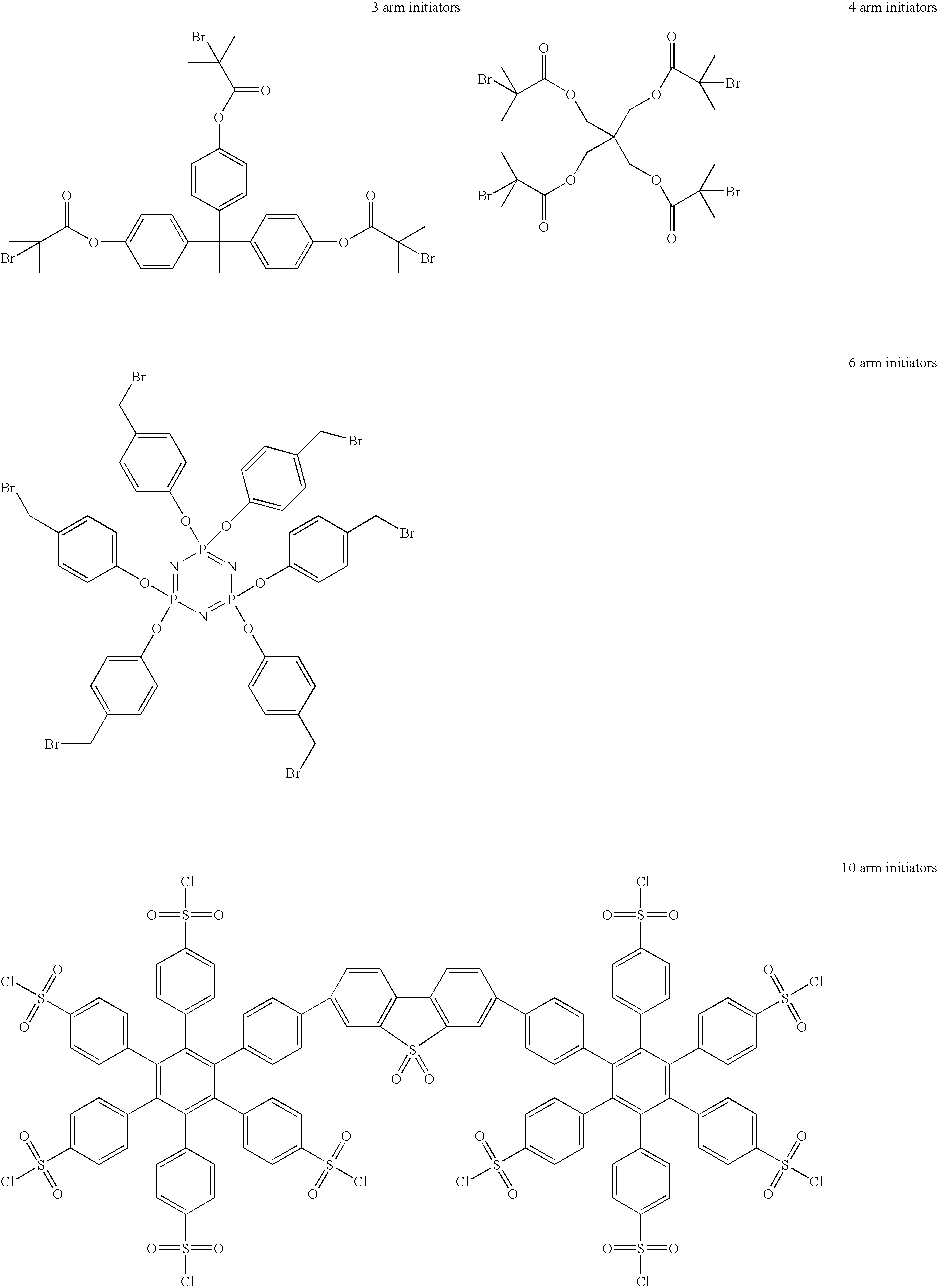

This method widens the flexibility from various angles and may prove an excellent synthetic procedure for different permutation and combinations for generating RPM.

The hydrophilic core can be composed of either homo- or copolymers of hydroxyl-bearing (alkyl)acrylates. Indeed, copolymers of hydroxyl-bearing (alkyl)acrylate with an ionisable (alkyl)acrylate derivative (acidic or basic) can also be used in the preparation of RPM.

In the present invention, RPM are obtained from a 4-arm ATRP initiator which is then used in the polymerization of hydroxyl-bearing (alkyl)acrylate derivatives to generate amphiphilic star-shaped polymers. These polymers present a hydrophilic center core with a hydrophobic periphery. Two synthetic routes have been proposed in order to obtain amphiphilic star shaped polymers (Scheme 1).

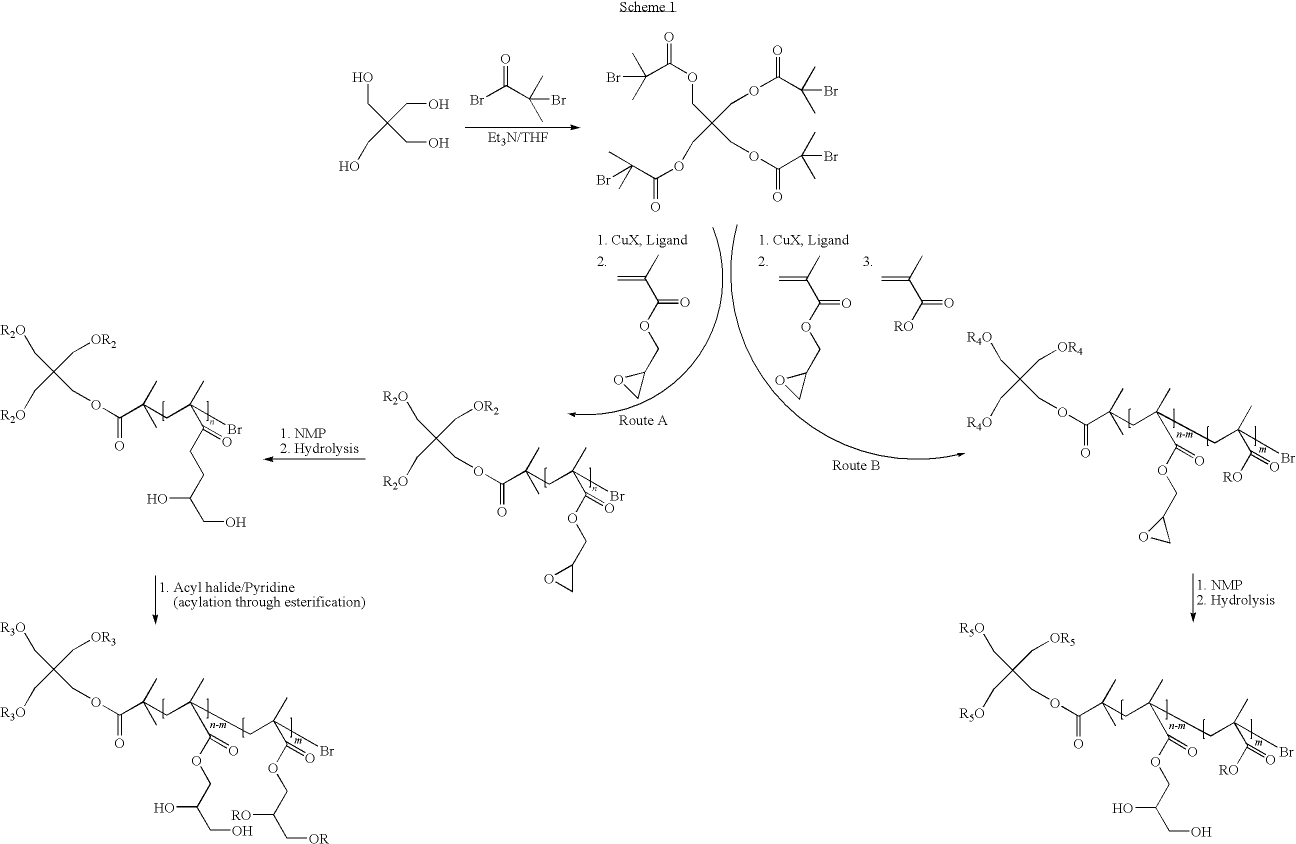

The first approach (route A) involves the synthesis of a hydrophilic poly(glycerol (alkyl)acrylate) polymer, which is then chemically modified to introduce the hydrophobic segment. The hydrophobicity of the micelle can be tailored by varying the length of the hydrocarbon chain or the extent of chemical modification. In Scheme 1, for route A and route B, bromine derivatives have been used but they can be replaced by any other suitable halogen derivatives. However, bromine derivatives are preferred.

For example, star-shaped polymers have been obtained from the ATRP polymerization and subsequent hydrolysis of poly(glycidylmethacrylate) to yield star-shaped poly(glycerolmethacrylate).

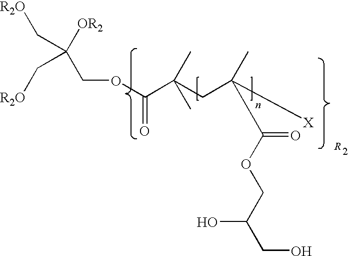

R2 represents the repeating unit at the other three arms, X represents a halogen atom (preferably a bromine atom or a chlorine atom); n represents the number of glycerol methacrylate units in poly(glycerolmethacrylate) and it has a value of 20 to 3000. Alternatively, it can have a value of 20 to 2730.

The hydrophilic polymer obtained in the second step, is then modified to introduce the hydrophobic outer shell through acylation of pendant hydroxyl groups using acylating agents such as fatty acids derivatives (e.g. stearoyl chloride, palmitoyl chloride or various similar derivatives). The general formula I can be represented as follows:

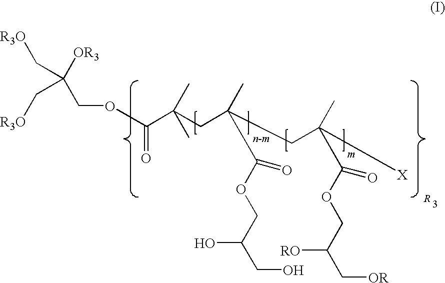

(n-m) represents the number of monomer unit in the hydrophilic segment and m represents the number of hydrophobic unit in the RPM. Preferably, m is an integer having a value from 3 to 1500 and n is an integer having a value from 20 to 3000. Alternatively, m can be an integer having a value from 4 to 1330 and n an integer having a value from 20 to 2730.

R₃ represents the repeating unit for each arm and R can represent a C₄-C₃₀ (preferably C₈-C₂o) acyl or alkyl chain, or a hydrogen atom. The alkyl chain and the acyl chain can be linear or branched, saturated or unsaturated. When a chain is unsaturated, it can be monounsaturated or polyunsaturated. R can be a fatty acid (preferably a C4-C20 fatty acid), a derivative thereof or a hydrogen atom, which is connected to the oxygen atom of the hydroxyl groups of the polyol. The fatty acid can be connected to a hydroxyl group via its carbonyl, thereby forming an ester bond. It will be understood that the % of grafting of the polyols of the present invention will be determined on the basis of the hydroxyl groups that are connected to one of the previously mentioned chains. A high grafting percentage will result in a low percentage of free hydroxyls groups (unalkylated or unacylated). The person skilled in the art would thus understand that for each unit between the brackets, which is repeated “m” times both R can be the same acyl or alkyl chain, one R can be a hydrogen atom and the other R can be an acyl or alkyl chain, or both R can be a hydrogen atom. There are thus three possible types of units in the “m” units. These three types respectfully comprise 0, 1 and 2 free hydroxyl groups. They can be called ml, m2 and m3, respectively. The value of m can be equal to the sum of ml, m2, and m3 (m=ml+m2+m3). The nature of the various R groups present in the polyol will thus vary in accordance with the grafting ratio. In accordance with the grafting ratio, the amount of each ml, m2 and m3 units can be distributed substantially randomly.

X represents a halogen atom and preferably Br or Cl.

In the second approach (route B), amphiphilic star-shaped structures are obtained from copolymerization of glycerol (alkyl)acrylate and alkyl(alkyl)acrylate derivatives. The properties of the RPM can easily be tailored by adjusting the degree of polymerization of each constituents. Synthesis via this route, gives unlimited choice of incorporating the hydrophobic region in a well controlled fashion. In the present invention, amphiphilic copolymers are prepared by the sequential polymerization of glycidyl methacrylate and an alkyl(alkyl)acrylate (e.g. lauryl methacrylate). The general formula (II) can be represented as follows:

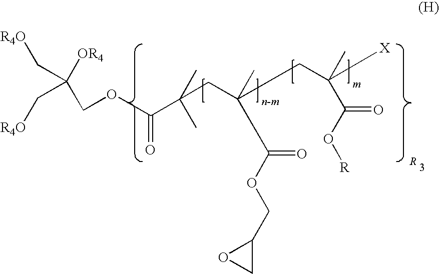

R4 is the repeating unit of each arm. R can be a C1-C30 alkyl chain or a hydrogen atom. The alkyl chain can be linear or branched, saturated or unsaturated. When a chain is unsaturated, it can be monounsaturated or polyunsaturated. R can also be of formula CpH2p+1 where p has a value from 1 to 30 and preferably 1 to 20. By preparing various compounds of formula II having different value for the R group, it is possible to make a library of compounds with different alkyl chain lengths.

Preferably, m is an integer having a value from 3 to 1500 and n is an integer having a value from 20 to 3000.

As previously mentioned, the % of grafting of the polyols of the present invention will be determined on the basis of the hydroxyl groups that are connected to one of the previously mentioned alkyl chains.

Glycidyl methacrylate is then quantitatively hydrolyzed to yield amphiphilic star-shaped poly(glycerolmethacrylate)-b-poly(alkyl(alkyl)acrylate) polymers of formula (III).

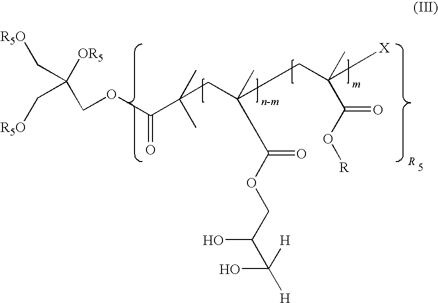

R₅ is the repeating unit on each arm, R, X m and n are as previously defined for formula (II).

The person skilled in the art would thus understand that for each unit between the brackets, which is repeated “m” times the R can be an alkyl chain or a hydrogen atom. Therefore, there will be some units in which R is an alkyl chain and some in which R is an hydrogen atom. Therefore for each unit of the m units of the polyol, the R group can independently be an alkyl chain or a hydrogen atom. There are thus two possible types of units in the “m” units. These two types respectfully comprise 0 and 1 free hydroxyl groups. They can be called ml, and m2, respectively. The value of m can be equal to the sum of ml and m2 (m=ml+m2). The nature of the various R groups present in the polyol will thus vary in accordance with the grafting ratio. In accordance with the grafting ratio, the amount of each ml and m2 units can be distributed substantially randomly.

The resulting amphiphilic polymers can form RPM in organic solvents (e.g. methylene chloride) and various oils (e.g. soybean or corn oil). Such RPM can accommodate hydrophilic guest and increase their solubility in apolar environments.

### EXAMPLES

The invention will now be illustrated by, but is not intended to be limited to, the following examples.

Materials:

All products were purchased from Aldrich (Milwaukee, Wis.). Copper(I) bromide (99.99% Grade), 1-methyl-2-pyrrolidinone, 2-bromoisobutyryl bromide, anhydrous triethylamine, anhydrous pyridine, anhydrous toluene, stearoyl chloride, palmitoyl chloride, myristoyl chloride and bipyridyl were used without further purification. Glycidylmethacrylate and laurylmethacrylate were used as monomers. Prior to use, tetrahydrofuran (THF) was distilled over sodium, using benzophenone as drying indicator.

In the following examples the polymers will be referred to as follows:

PGMA_{X}: poly(glycidylmethacrylate) where x is the degree of polymerization.

PGO_{H}MA_{Z}: poly(glycerol methacrylate) where z is the degree of polymerization of glycerol methacrylate.

PGo_{├}iMA-ib-PLMA (x:y): block copolymers of glycerol methacrylate and laurylmethacrylate. The numbers in parenthesis refer to the ratio of glycerolmethacrylate to laurylmethacrylate.

PG_{0 H}MA-CY (grafting ratio N %): acylated poly(glycerolmethacrylate) where Y represents the number of carbon atoms in the acyl group. The number in parenthesis refers to the number of —OH equivalents that have been grafted or acylated.

### Example 1

### Synthesis of ATRP initiator: Tetrakis(2-bromoisobutyryl) pentaerythritolate

2-bromoisobutyryl bromide (24.7 ml_{—}, 0.2 mol) was slowly added to a slightly cooled solution of pentaerythritol (3.4 g, 0.025 mol) and triethylamine (21 ml_{—}, 0.1 5 mol) in anhydrous THF (85 ml_). The solution was then warmed to room temperature and stirred for 24 h. The mixture was poured into water and extracted with methylene chloride. The organic extracts were washed successively with 1M HCl and 1M NaOH (containing NaCl), and dried over magnesium sulfate. The solvent was removed under reduced pressure. The product was recrystallized in ethanol/diethyl ether. The title compound was recovered as white crystals by simple filtration. Yield: 95%. This radical initiator is very stable in presence of air or water. ¹H— NMR (δ, ppm, CDCl₃): 4.33 (s, 8H₁—CH₂); 1.94 (s, 24H, —CH₃).

### Example 2

### ATRP Synthesis of Star-Shaped Poly(Glycidylmethacrylate): (PGMA-io₆)

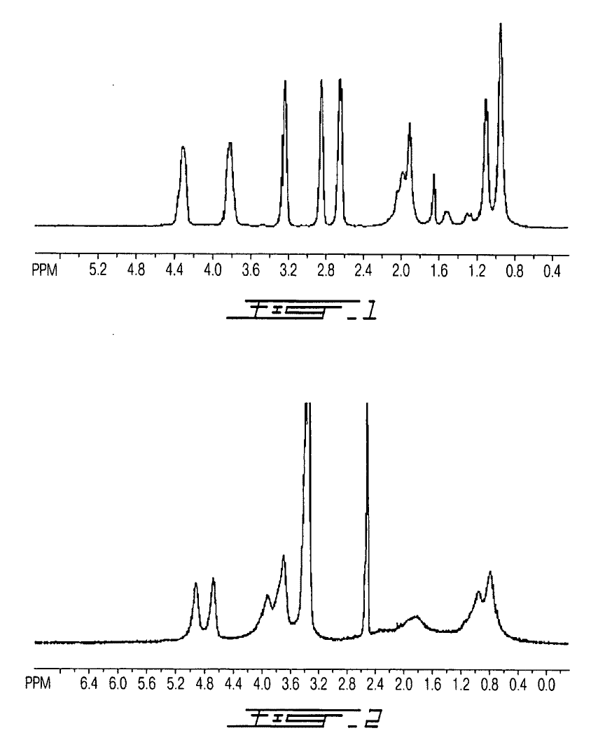

A 1000 ml_ round bottom flask was loaded with ATRP tetra initiator (1 eq., 1.0 mM), bipyridyl (1 eq., 0.0015 mol) and glycidylmethacrylate (15 ml_{—}, 106 eq.). The mixture was degassed and kept under inert atmosphere. THF (350 ml_) was added and stirred for 10 min. to homogenize the solution, which was followed by addition of Cu(I)Br. The mixture was heated to 9O ° C. and the reaction was run for 30 h. After cooling down, the mixture was passed through a silica gel column using THF as the eluent to remove copper bromide. The solvent was evaporated and the polymer was precipitated twice in ether. The compound was further purified by soxhlet extraction using ether. Excess solvent was removed under reduced pressure. Yield: 75-85%. Mn=15,000 g/mol, polydispersity index: 1.34 FIG. 1 shows the NMR spectrum of PGMA₁₀₆.

### Example 3

### Synthesis of polyfølycerolmethacrylate): (PGOHMA-IO6)

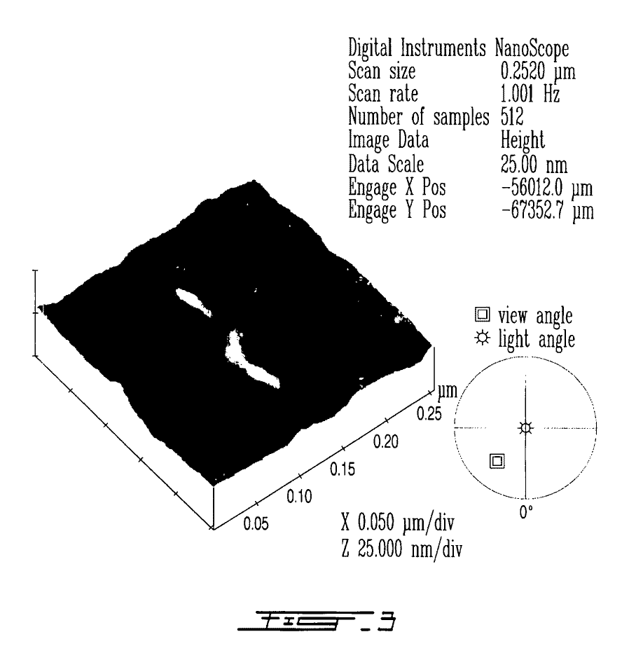

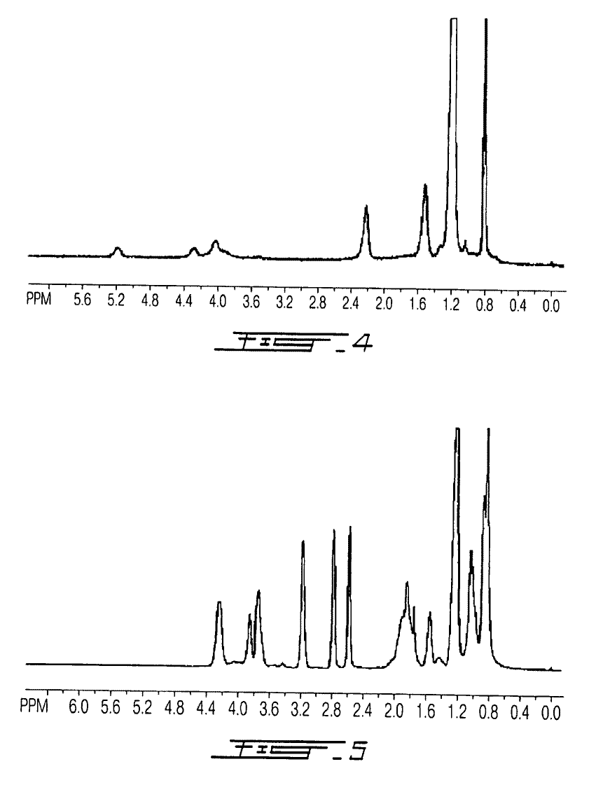

Poly(glycerol methacrylate) (PG_{O}HMA) is obtained through the hydrolysis of the epoxy ring of PGMA. In a typical procedure, PGMA (6 g, 0.4 mM, number-average molecular weight (Mn=14,000 g/mol)) was dissolved in 1-methyl-2-pyrrolidinone (72 mL, NMP) under gentle stirring. After complete dissolution of the polymer, 25 mL of water (20 eq.) was added drop wise and the mixture was left to react at 120° C. for 24 h. The hydrolyzed polymer was dialyzed against water for 48 h and then freeze-dried. Yield: 50-60%. FIG. 2 shows the ¹H-NMR spectrum of the compound. FIG. 3. demonstrates the AFM picture of poly(glycerolmethacrylate): (PG_{O}HMA; Mn: 150,000 g/mol).

### Example 4

### Synthesis of stearoyl substituted poly(glycerolmethacrylate): (PGOHMA-C18) (grafting ratio 50%)

PGOHMA (0.2 g, 0.0024 mol of hydroxyl group) was dried by azeotropic distillation and solubilized in pyridine (30 mL) in the presence of catalytic amounts of 1-methylimidazole. Stearoyl chloride (0.41 mL, 0.0012 mol) dissolved in toluene was slowly added to the reaction mixture under anhydrous and inert conditions. The reaction was left to proceed overnight under reflux. Following completion of the reaction, 5 g of K₂CO₃ was added for work-up. Pyridine was removed under reduced pressure. Residues were removed by azeotropic distillation. The crude compound was dissolved in chloroform and dialyzed against chloroform for 24 h. The solvent was removed under reduced pressure to yield the acylated polymer as brownish waxy flakes. FIG. 4 shows the NMR spectrum of the compound. The yield of the product was 64%.

### Example 5

### Synthesis of diblock copolymers poly(glycidylmethacrylate)-b/oc/c-poly(laurylmethacrylate): (PGMA-fo-PLMA) (75:25)

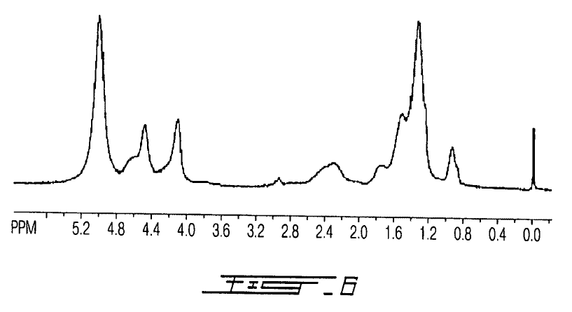

Diblock copolymers were prepared by the sequential polymerization of glycidylmethacrylate and laurylmethacrylate. Glycidyl methacrylate (5 mL, 0.036 mol, 1 eq) was polymerized first according to the procedure described previously. The reaction was carried out for 26 h at which point laurylmethacrylate (2.68 mL, 0.25 eq.) was added to the reaction pot. The mixture was left to further react for 24 h at 90° C. The resulting polymer was filtered through a silica gel column using THF as the eluent to remove copper bromide. The solvent was evaporated and polymer was precipitated twice in diethylether. The compound was further purified by soxhlet extraction using ether. Excess solvent was removed under reduced pressure. Yield: 86%. The polymer was dried and characterized by NMR as shown in FIG. 5.

### Example 6

### Synthesis of poly(glycerolmethacrylate)-b/oc/f-poly(laurylmethacrylate): (PGo_{H}MA-b-PLMA) (75:25)

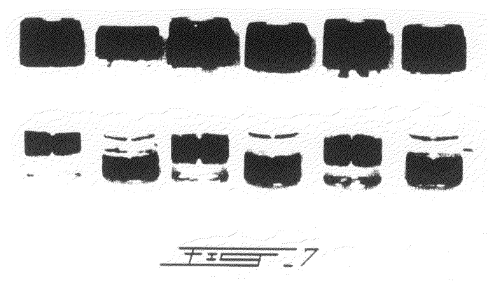

The polymer of example 5 (PGMA-b-LMA (75:25)) (6 g) was dissolved in 1-methyl-2-pyrrolidinone (72 mL) under gentle stirring. After complete dissolution of the polymer, 15 mL of water was added dropwise and the mixture was left to react at 120° C. for 24 h. The hydrolyzed polymer was dialyzed against water for 48 h and then freeze-dried. Yield: 60-70%. FIG. 6 shows the ¹H NMR spectrum of the compound.

### Example 7

### Particle Size Analysis

Solutions of acylated poly(glycerol methacrylate) were prepared in methylene chloride at varying concentrations (1 to 10 g/L). Particle size was determined by dynamic light scattering at 25° C. and at a 90° angle. Aggregates of 100 and 400 nm were observed for acylated star-shaped PGO_{H}MA-C16 (grafting ratio 25%) and PG_{O}HMA-C16 (grafting ratio 60%), respectively.

### Example 8

### Solubility in Oils

The solubility of acylated star-shaped poly(glycerol methacrylate) was evaluated by dissolving the polymer in increasing amounts of pharmaceutical oils until a clear solution was obtained. PG_{O}HMA-C16 (grafting ratio 60%), was found to be soluble in corn oil and soybean oil (5 mg/ml_).

### Example 9

### Extraction of a Hydrophilic Compound from Water into Methylene Chloride

Aqueous solutions of an anionic dye (Congo Red) were prepared (1 ml_ of 0.025 to 5 mg/mL solutions) and gently mixed with solutions of PGO_{H}MA-C16 (grafting ratio 25%) in methylene chloride (1 ml_{—}; 5 mg/mL). The aqueous phase was assayed by absorbance (λ=500 nm) following complete separation of the two phases. The amount of dye extracted was determined as the difference between the initial and remaining amount of dye in water. PG_{OH}MA-C16 (grafting ratio 25%) was able to extract 50-80% of the dye from water into methylene chloride (FIG. 7) corresponding to a maximal dye concentration of 12% (w/w) in polymeric micelles.

### Example 10

### Extraction of a Therapeutic Peptide from Water into Methylene Chloride

1-mL of an aqueous solution of vasopressin (spiked with ³H-vasopressin) (3.3 mg/mL; 25% (w/w) vs RPM) was gently mixed with a solution of PG_{O}HMA-C16 (grafting ratio 25%) in methylene chloride (1 mL; 10 mg/mL). Following complete phase separation, both the aqueous and organic layers were assayed for radioactivity. PG_{OH}MA-C16 (grafting ratio 25%) was able to partially extract vasopressin from water into methylene chloride corresponding to a peptide concentration of 0.2% (w/w) in polymeric micelles. In the absence of polymer, no significant amount of peptide is assayed in the organic phase. This example shows that in the presence of RPM, the peptide is partially retained and solubilized in the organic phase.

### Example 11

### Extraction of a Therapeutic Peptide (Vasopressin) from Methylene Chloride into Simulated Gastric Buffer

556 μl_ of a solution of vasopressin (spiked with ³H-vasopressin) in ethanol (1 mg/mL; 10% (w/w) vs RPM) was mixed with a solution of PG_{O}HMA-C16 (grafting ratio 25%) in methylene chloride (500 μL; 10 mg/mL). Following complete evaporation of the solvents, the polymer-peptide complexes were re-dissolved in methylene chloride (1 ml_). The aqueous phase (1 mL of non-enzymatic simulated gastric buffer; pH 1.2) was then added. The two phases were gently mixed together for 24 h. Following complete phase separation, both the aqueous and organic layers were assayed for radioactivity. Controls without polymer were also prepared. In the presence of the polymer, 10% of the peptide was retained in the organic phase corresponding to a peptide concentration of 1% (w/w) in polymeric micelles while for the controls almost no radioactivity was found in methylene chloride. This example shows that a significant portion of the peptide can be solubilized and partially retained in an organic phase in the presence of the polymer.

### Example 12

### Extraction of a Therapeutic Peptide (Vasopressin) from Soybean Oil into Water

The polymer (PG_{O}HMA-C16 (grafting ratio 60%)) was dissolved in methylene chloride (1 mL; 5 mg/mL) and mixed with a solution of vasopressin (spiked with ³H-vasopressin) in ethanol (0.05 mL; 1 mg/mL). Soybean oil (500 μL) was added to the mixture. The aqueous phase (phosphate buffer, pH 7.2, 500 μL) was added after complete removal of the organic solvents. The two phases were gently mixed together for 24 h. Following complete phase separation, both the aqueous and soybean oil layers were assayed for radioactivity. Controls without polymer were also prepared and analyzed. In the presence of PGO_{H}MA-C16 (grafting ratio 60%), over 60% of the radioactivity was found in the organic phase, whereas the vasopressin was almost completely extracted from the controls. This example shows that the polymer allows for the solubilization of the peptide in an oleaginous phase while providing sustained release in an aqueous medium.

### Example 13

### Release of Hydrophilic Compound (Vasopressin) from Reverse Micelles

The polymer PGOHMA-C12 (grafting ratio 60%) was dissolved in methylene chloride (1 ml_{—}; 5 mg ml_{—}⁻¹) and mixed with 51 μl_ of an ethanolic solution of vasopressin (1 gl_{—}⁻¹; spiked with ³H-vasopressin). Ethyl oleate (250 μl_) was added to the mixture. Following the complete evaporation of the volatile organic solvents, 50 mg of the micellar solution in ethyl oleate were emulsified in water (440 μl_) in the presence of polysorbate 80 as an emulsifier (10 mg). The resulting emulsion was loaded inside a dialysis membrane and dialyzed against a phosphate buffer (200 ml_; NaH₂PO4: 0.06 M; NaOH 0.015 M; pH 6.8) containing 0.2% (w/w) of polysorbate 20 to prevent non-specific adsorption. Aliquots of the release media were taken at pre-determined time points and replaced by fresh medium. Vasopressin content was determined by radioactivity counting. The control consisted in ethyl oleate emulsified in an aqueous peptide solution. After 6 h, over 60% of the peptide was released from the micellar formulation. This example shows the loaded hydrophilic compounds can be released following emulsification of the oleaginous micellar phase.

### Example 14

### Extraction of a Therapeutic Peptide (Vasopressin) from Water into Ethyl Oleate

The polymer (PGO_{H}MA-C18 (grafting ratio 60%) is dissolved in dichloromethane (5 mg/mL) and mixed with a solution of vasopressin (spiked with radiolabeld vasopressin) in ethanol (1 mg/mL; 51 μl_). Ethyl oleate (500 μl_) is added to the mixture. Following complete removal of the organic solvents, the aqueous phase is added. The two phases are gently mixed together for 24 h. Following complete phase separation, both the aqueous and organic layers were assayed for radioactivity. Controls without polymer were also prepared. In the presence of PGO_{H}MA-C18 (grafting ratio 60%), over 80% of the radioactivity was found in the organic phase.

All the documents that are referred to in the present document are hereby incorporated by reference.

While the invention has been described with particular reference to the illustrated embodiment, it will be understood that numerous modifications thereto will appear to those skilled in the art. Accordingly, the above description and accompanying drawings should be taken as illustrative of the invention and not in a limiting sense.

1. A reverse micelle composition comprising:
a) a reverse micelle comprising a hydrophilic core consisting of a star-shaped polyol, and a hydrophobic shell comprising alkyl chains, said alkyl chains being the same or different; and
b) a water-soluble active agent contained in said hydrophilic core.

2. The composition of claim 1, wherein said star-shaped polyol is selected from the group consisting of poly(glycerol (alkyl)acrylate), poly(hydroxy(alkyl) (alkyl)acrylate), poly(β-methylglucoside (alkyl)acrylate), poly(2-gluconamidoethyl (alkyl)acrylate) and poly(2-lactobionamidoethyl (alkyl)acrylate.

3. (canceled)

4. (canceled)

5. The composition of claim 1, wherein said polyol is a copolymer of hydroxyl-bearing (alkyl)acrylate and an ionisable (alkyl)acrylate derivative.

6. (canceled)

7. The composition of claim 1, wherein said star-shaped polymer is formed using a multifunctional initiator bearing between 3 to 20 initiating arms.

8. (canceled)

9. (canceled)

10. The composition of claim 1, wherein each of said alkyl chains is linked to the star-shaped polyol core by means of a covalent bond.

11. The composition of claim 1, wherein each of said alkyl chains is linked to the star-shaped core via a non-hydrolysable bond.

12. (canceled)

13. The composition of claim 12, wherein each of said alkyl chains is directly connected to a hydroxyl group of the polyol through said ether bond.

14. The composition of claim 1, wherein each of the alkyl chains is linked to the star-shaped core via a hydrolysable bond.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. The composition of claim 1, wherein said polyol is a copolymer poly(glycerolmethacrylate)-block-poly(laurylmethacrylate).

28. The composition of claim 1, wherein the star-shaped polyol, excluding said alkyl chains, has a molecular weight between 3000 and 200000 g/mol.

29. The composition of claim 1, wherein said star-shaped polyol is of formula (I): 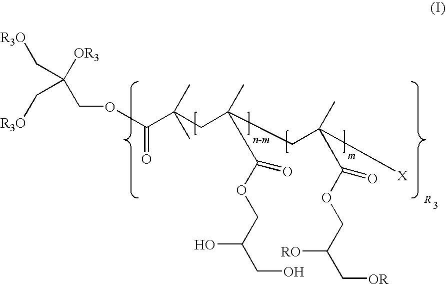
wherein
X represents a halogen atom;
each of said R is independently a C₄-C₃₀ alkyl group, a C₄-C₃₀ acyl group, or a hydrogen atom;
R₃ represents a repeating unit for each arm of said star-shaped polyol;
m is an integer having a value from 3 to 1500; and
n is an integer having a value from 20 to 3000.

30. (canceled)

31. (canceled)

32. The composition of claim 1, wherein said star-shaped polyol is of formula (III): 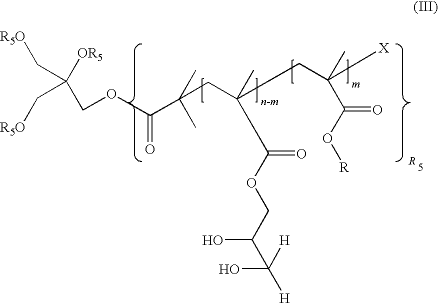
wherein
X represents a halogen atom;
each of said R is independently a C₁-C₃₀ alkyl group, or a hydrogen atom;
R₅ represents a repeating unit for each arm of said star-shaped polyol;
m is an integer having a value from 3 to 1500; and
n is an integer having a value from 20 to 3000.

33. (canceled)

34. The composition of claim 1, wherein said active agent is selected from the group consisting of cytokines, peptidomimetics, peptides, proteins, toxoids, antibodies, nucleosides, nucleotides, nucleic acids, polysaccharides, analgesics and anti-inflammatory agents, anthelmintics, anti-arrhythmic agents, anti-asthma agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents and immunosuppressants, anti-protozoal agents, anti-thyroid agents, anti-tussives, anxiolytic, sedatives, hypnotics and neuroleptics, β-blockers, cardiac inotropic agents, diuretics, anti-parkinsonian agents, gastrointestinal agents, histamine H,-receptor antagonists, keratolytics, lipid regulating agents, muscle relaxants, anti-anginal agents, nutritional agents, analgesics, sex hormones, stimulants, and cytokines.

35. A pharmaceutical composition which comprises a reverse micelle composition as defined in claim 1, dissolved in a pharmaceutically acceptable oil.

36. (canceled)

37. A cosmetic preparation which comprises a reverse micelle composition as defined in claim 1, dissolved in a pharmaceutically acceptable oil.

38. A process for preparing the pharmaceutical composition of claim 35, which comprises the dissolution of a hydrophilic active ingredient and an amphiphilic star-shaped polymer in an oleaginous vehicle, with or without surface active agents.

39. (canceled)

40. A reverse micelle composition comprising:
a reverse micelle comprising a star-shaped polyol at least partially grafted with alkyl chains or acyl chains, said alkyl chains being the same or different and said acyl chains being the same or different, wherein an inner portion of said star-shaped polyol comprising free hydroxyl groups defines a hydrophilic core, and wherein an outer portion of said star-shaped polyol comprising said alkyl chains or said acyl chains defines a hydrophobic shell; and
a water-soluble active agent contained in said hydrophilic core.

41. The composition of claim 40, wherein said star-shaped polyol is selected from the group consisting of poly(glycerol (alkyl)acrylate), poly(hydroxy(alkyl) (alkyl)acrylate), poly(β-methylglucoside (alkyl)acrylate), poly(2-gluconamidoethyl (alkyl)acrylate) and poly(2-lactobionamidoethyl (alkyl)acrylate.

42. (canceled)

43. (canceled)

44. (canceled)

45. (canceled)

46. The composition of claim 40, wherein said star-shaped polymer is formed using a multifunctional initiator bearing from 3 to 20 initiating arms.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The composition of claim 40, wherein said star-shaped polyol is of formula (I): 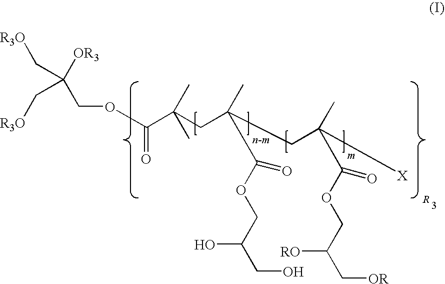
wherein
X represents a halogen atom;
each of said R is independently a C₄-C₃₀ alkyl group, a C₄-C₃₀ acyl group, or a hydrogen atom;
R₃ represents a repeating unit for each arm of said star-shaped polyol;
m is an integer having a value from 3 to 1500; and
n is an integer having a value from 20 to 3000.

64. The composition of claim 63, wherein each of said R is independently a C₄-C₂₀ acyl group or a hydrogen atom.

65. (canceled)

66. The composition of claim 40, wherein said star-shaped polyol is of formula (III): 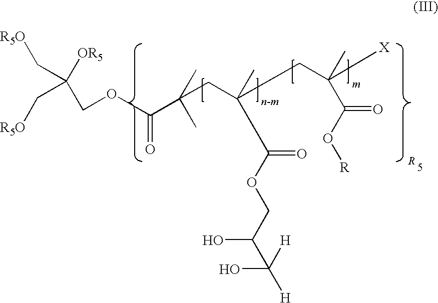
wherein
X represents a halogen atom;
each of said R is independently a C₁-C₃₀ alkyl group, or a hydrogen atom;
R₅ represents a repeating unit for each arm of said star-shaped polyol;
m is an integer having a value from 3 to 1500; and
n is an integer having a value from 20 to 3000.

67. (canceled)

68. The composition of claim 40, wherein at least 20% of the hydroxyls groups of said polyol are grafted with said acyl chains or said alkyl chains.

69. (canceled)

70. (canceled)

71. (canceled)

72. (canceled)

73. (canceled)

74. A pharmaceutical composition which comprises a reverse micelle composition as defined in claim 40, dissolved in a pharmaceutically acceptable oil.

75. (canceled)

76. (canceled)

77. A process for preparing the pharmaceutical composition of claim 74, which comprises the dissolution of a hydrophilic active ingredient and an amphiphilic star-shaped polymer in an oleaginous vehicle, with or without surface active agents.

78. (canceled)

79. A process for preparing a reverse micelle composition as defined in claim 1 comprising mixing together said water-soluble active agent and a composition comprising a micelle as defined in claim 1 and a hydrophobic pharmaceutically acceptable vehicle.

80. (canceled)

81. The process of claim 79, wherein said hydrophilic active agent, said micelle and said pharmaceutically acceptable vehicle are mixed together and heated.

82. A process for preparing a reverse micelle composition as defined in claim 1 comprising mixing together a first composition comprising a water-soluble active agent and a polar solvent, and a second composition comprising a micelle as defined in claim 1 and a hydrophobic pharmaceutically acceptable vehicle.

83. (canceled)

84. The process of claim 82, wherein said hydrophilic active agent, said polar solvent, said micelle and said pharmaceutically acceptable vehicle are mixed together and heated.

85. The process of claim 82, further comprising separating said polar solvent from the rest of said micelle composition.

86. (canceled)

87. (canceled)

88. (canceled)

89. (canceled)

90. (canceled)

91. A delivery system for a hydrophilic active agent, said system comprising a hydrophilic core consisting of a star-shaped polyol and being adapted to receive the hydrophilic active agent; and a hydrophobic shell consisting of alkyl chains, said alkyl chains being the same or different.

92. (canceled)

93. (canceled)

94. (canceled)

95. (canceled)

96. (canceled)

97. (canceled)

98. (canceled)

99. (canceled)

100. (canceled)

101. A delivery system for a hydrophilic active agent, said system comprising a star-shaped polyol at least partially grafted with alkyl chains or acyl chains, said alkyl chains being the same or different and said acyl chains being the same or different, wherein an inner portion of said star-shaped polyol comprises free hydroxyl groups defining a hydrophilic core, and wherein an outer portion of said star-shaped polyol comprises said alkyl chains or said acyl chains defining a hydrophobic shell.

102. (canceled)

103. (canceled)

104. (canceled)

105. (canceled)

106. The delivery system of claim 101, wherein at least 20% of the hydroxyls groups of said polyol are grafted with said acyl chains or said alkyl chains.

107. (canceled)

108. (canceled)

109. (canceled)

110. (canceled)

111. (canceled)

112. (canceled)

113. The delivery system of claim 91, wherein said star-shaped polyol is selected from the group consisting of poly(glycerol (alkyl)acrylate), poly(hydroxy(alkyl) (alkyl)acrylate), poly(β-methylglucoside (alkyl)acrylate), poly(2-gluconamidoethyl (alkyl)acrylate) and poly(2-lactobionamidoethyl (alkyl)acrylate.

114. (canceled)

115. (canceled)

116. (canceled)

117. (canceled)

118. The delivery system of claim 91, wherein said star-shaped polymer is formed using a multifunctional initiator bearing between 3 to 20 initiating arms.

119. (canceled)

120. (canceled)

121. (canceled)

122. (canceled)

123. (canceled)

124. (canceled)

125. (canceled)

126. (canceled)

127. (canceled)

128. The delivery system of claim 91, wherein said star-shaped polyol is of formula (I): 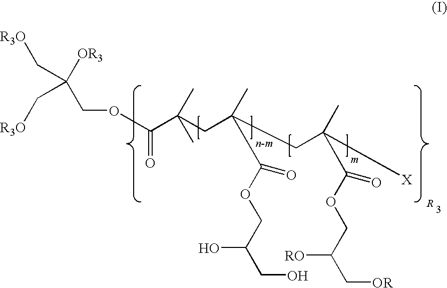
wherein
X represents a halogen atom;
each of said R is independently a C₄-C₃₀ alkyl group, C₄-C₃₀ acyl group, or a hydrogen atom;
R₃ represents a repeating unit for each arm of said star-shaped polyol;
m is an integer having a value from 3 to 1500; and
n is an integer having a value from 20 to 3000.

129. (canceled)

130. (canceled)

131. The delivery system of claim 91, wherein said star-shaped polyol is of formula (III): 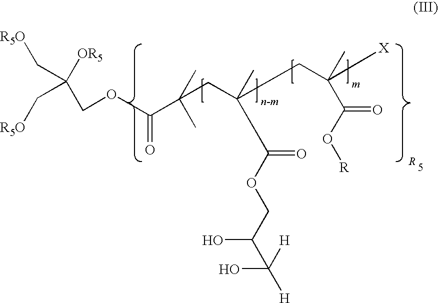
wherein
X represents a halogen atom;
each of said R is independently a C₁-C₃₀ alkyl group, or a hydrogen atom;
R₅ represents a repeating unit for each arm of said star-shaped polyol;
m is an integer having a value from 3 to 1500; and
n is an integer having a value from 20 to 3000.

132. (canceled)

133. (canceled)

134. (canceled)

135. (canceled)

136. (canceled)
